(19) United States
(12) Patent Application Publication
(10) Pub. No.: US 2025/0308660 A1
(43) Pub. Date: Oct. 2, 2025

(54) COMPUTER SYSTEM AND DATA ANALYSIS METHOD

(71) Applicant: Hitachi High-Tech Corporation, Tokyo (JP)

(72) Inventors: Yasuaki NAKAMURA, Tokyo (JP); Wataru TAKEUCHI, Tokyo (JP); Takashi SHIMIZU, Tokyo (JP); Takeshi ISHIZAKI, Tokyo (JP)

(21) Appl. No.: 18/823,795

(22) Filed: Sep. 4, 2024

(30) Foreign Application Priority Data
Mar. 26, 2024 (JP) 2024-049835

(51) Int. Cl. G16H 20/00; G06N 20/20; G16H 50/70

(52) U.S. Cl. CPC G16H 20/00; G06N 20/20; G16H 50/70

(57) ABSTRACT

A computer system is accessibly connected to a database that stores data including values of a plurality of factors. The computer system repeatedly executes: first processing of partitioning an analysis data set including a plurality of pieces of data into a first data set and a second data set; second processing of searching, using the first data set, for a branching condition for partitioning the analysis data set into two groups, evaluating an intervention effect using the second data set, determining the branching condition to be used, and generating a decision tree that includes at least one branching condition and is used to predict an event; and third processing of calculating a score indicating quality of a branch of the decision tree for each of a plurality of decision trees. The computer system generates information for displaying the plurality of decision trees and the score.

### CLAIM OF PRIORITY

The present application claims priority from Japanese patent application JP 2024-049835 filed on Mar. 26, 2024, the content of which is hereby incorporated by reference into this application.

### BACKGROUND OF THE INVENTION

### 1. Field of the Invention

The present invention relates to a system and a method for analyzing data.

### 2. Description of Related Art

Conventional medical practice has promoted standardization and guideline creation based on randomized controlled trials, and on the other hand, it has become evident that a treatment is not effective for all patients and there is individual variability. Therefore, current medical practice focuses on pursuit of an optimal treatment selection tailored to an individual characteristic of a patient. For example, a comprehensive medical data analysis system has been disclosed in which patients are classified into subtypes (stratification) based on patient characteristics and the like, and treatments and outcomes for similar patients are analyzed (see JP 2017-502439 A).

The comprehensive medical data analysis system includes a medical main server including an intelligent medical engine, the intelligent medical engine is communicably coupled to a central database that is a confidential electronic medical record database, and is further communicably coupled to a hospital, a clinic, and other medical resources via a network. The intelligent medical engine receives a large number of medical records from potentially different countries, regions, and continents. The electronic medical records are provided from a hospital, a clinic, and other medical resources, and are supplied into the intelligent medical engine such that medical records of patients can be correlated by global large-scale analysis. The analysis is started by grouping (classifying) the medical records into subgroups of a plurality of levels according to a patient clinical parameter, a disease template, a treatment, and an outcome. When a new patient is input to the system, a parameter and a disease template of the patient are matched with a most similar subgroup for a possibly favorable outcome.

### CITATION LIST

### Patent Literature

PTL 1: JP2017-502439A

### Non Patent Literature

NPL 1: Athey, Susan, et al, “Recursive partitioning for heterogeneous causal effects” Proceedings of the National Academy of Sciences 113.27 (2016): 7353-7360.

### SUMMARY OF THE INVENTION

In general, a learning algorithm of a tree structure has a problem of overfitting. In order to prevent overfitting, a decision tree is generated by partitioning a data set that serves as a population into two data sets with different applications. Since the data set is partitioned randomly, a decision tree having a different structure is obtained with each training session.

Use of a random forest using a plurality of decision trees enables prediction of a treatment effect, but does not ensure interpretability (readability) of a prediction result.

The invention implements a system and a method for presenting a quantitative evaluation of prediction accuracy of a plurality of decision trees.

A representative example of the invention disclosed in the present application is as follows. That is, a computer system includes: a processor; and a storage apparatus connected to the processor, in which the computer system is accessibly connected to a database that stores data for evaluating an intervention effect, the data including values of a plurality of factors, and the processor repeatedly executes: first processing of partitioning an analysis data set including a plurality of pieces of the data into a first data set and a second data set; second processing of searching, using the first data set, for a branching condition for partitioning the analysis data set into two groups, the branching condition being defined by the factors and the values of the factors, evaluating the intervention effect using the second data set, determining the branching condition to be used, and generating a decision tree that includes at least one branching condition and is used to predict an event; and third processing of calculating a score indicating quality of a branch of the decision tree for each of a plurality of the decision trees, and generates and outputs information for displaying the plurality of decision trees and the score.

According to a representative aspect of the invention, a quantitative evaluation of prediction accuracy of a plurality of decision trees can be presented. Problems, configurations, and effects other than those described above will become apparent in the following description of embodiments.

### BRIEF DESCRIPTION OF THE DRAWINGS

FIG. 1 shows an example of outcomes of a prognostic factor and a predictive factor.

FIG. 2 shows an example of a method for partitioning a population.

FIG. 3 is a block diagram showing an example of a hardware structure of an analysis apparatus in a first embodiment.

FIG. 4 is a block diagram showing an example of a functional configuration of the analysis apparatus in the first embodiment.

FIG. 5 shows an example of a health care DB in the first embodiment.

FIG. 6 shows an example of patient data information in the first embodiment.

FIG. 7 shows an example of patient allocation information in the first embodiment.

FIG. 8 shows an example of an input screen presented by the analysis apparatus in the first embodiment.

FIG. 9 is a flowchart showing an example of analysis processing executed by the analysis apparatus in the first embodiment.

FIG. 10 shows an example of a decision tree generated by stratification processing executed by the analysis apparatus in the first embodiment.

FIG. 11 is a flowchart showing an example of the stratification processing executed by the analysis apparatus in the first embodiment.

FIG. 12A is a flowchart showing an example of branching condition search processing executed by the analysis apparatus in the first embodiment.

FIG. 12B is a flowchart showing an example of the branching condition search processing executed by the analysis apparatus in the first embodiment.

FIG. 13 is a flowchart showing an example of score calculation processing executed by the analysis apparatus in the first embodiment.

FIG. 14 is a flowchart showing an example of patient allocation processing executed by the analysis apparatus in the first embodiment.

### DESCRIPTION OF EMBODIMENTS

Hereinafter, an embodiment of the invention will be described with reference to the drawings. However, the invention is not to be construed as being limited to the description of the following embodiment. It will be easily understood by those skilled in the art that the specific configuration can be changed within a range not departing from the idea or spirit of the invention.

In the configurations of the invention to be described later, the same or similar configurations or functions are denoted by the same reference signs, and redundant description will be omitted.

Notations of “first”, “second”, “third”, and the like in the present specification and the like are used to identify the components, and the numbers and the order are not necessarily limited.

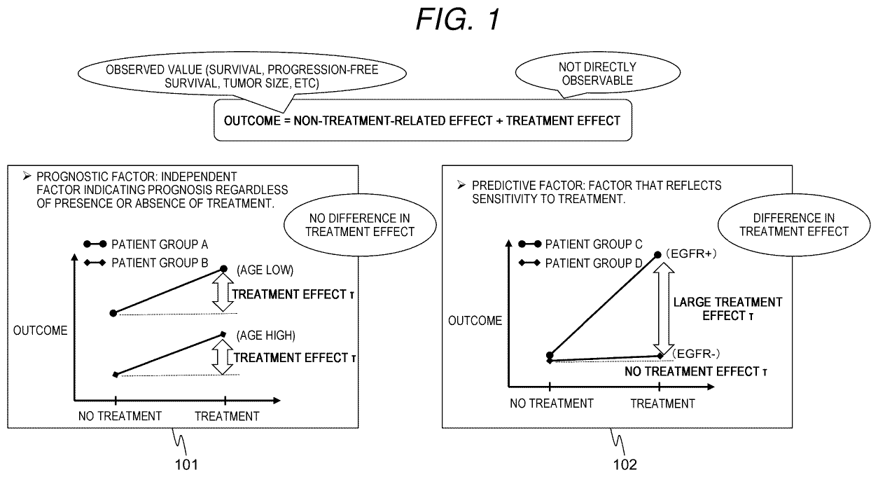

FIG. 1 shows an example of outcomes of a prognostic factor and a predictive factor.

An outcome is, for example, an observed value such as survival, progression-free survival, or a tumor size, and is a value inherently including a non-treatment-related effect and a treatment effect. The non-treatment-related effect and the treatment effect are not directly observable.

A graph 101 indicates the outcome before and after a treatment of patient groups A and B obtained by classifying a population of patients according to presence or absence of the prognostic factor. A graph 102 indicates the outcome before and after the treatment of patient groups C and D obtained by classifying the population of patients according to presence or absence of the predictive factor.

Each of the prognostic factor and the predictive factor is any factor in a factor group constituting a characteristic of a patient (hereinafter, referred to as a patient characteristic), and is a quantitative variable, that is, a covariate that varies with the outcome. The prognostic factor is an independent factor indicating prognosis regardless of presence or absence of the treatment, and is, for example, an age of the patient. The predictive factor is a factor that reflects sensitivity to the treatment, such as an epidermal growth factor receptor (EGFR), which is a factor showing different treatment effects depending on presence or absence of the predictive factor.

In the graph 101, the patient group A is a set (age low) of patients each having a low value of the prognostic factor indicating the age, and the patient group B is a set (age high) of patients each having a higher value of the prognostic factor indicating the age than the patient group A. In the graph 101, although the outcome before and after the treatment varies due to a difference between the patient groups A and B, there is no difference in a treatment effect τ (a difference in the outcome before and after the treatment) between the patient groups A and B.

In the graph 102, the patient group C is a set (EGFR+) of patients each having a large value of the predictive factor indicating EGFR, and the patient group D is a set (EGFR−) of patients each having a smaller predictive factor indicating EGFR than the patient group C. In the graph 102, the outcome before and after the treatment varies due to a difference between the patient groups C and D, and there is also a difference in the treatment effect τ (a difference in the outcome before and after the treatment) between the patient groups C and D. In the graph 102, the treatment effect τ of the patient group C is larger than the treatment effect τ of the patient group D.

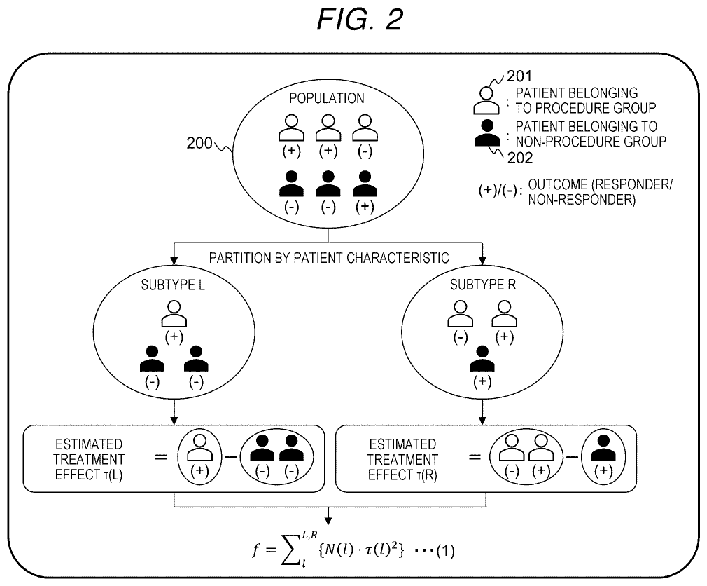

In this way, by partitioning the population of patients with the predictive factor such as EGFR, it is possible to support a treatment selection through a state classification by the treatment effect τ. When the population of patients is not partitioned with the predictive factor, it is possible to predict the treatment effect τ by a method shown in FIG. 2.

In the following description, partitioning of the population is also referred to as stratification.

FIG. 2 shows an example of the method for partitioning the population.

A population 200 includes a patient 201 belonging to a procedure group and a patient 202 belonging to a non-procedure group. The procedure group is a set of patients who receive a medical procedure for injury or illness, and the non-procedure group is a set of patients who receive no medical procedure for injury or illness. In addition, (+) indicates a responder and (−) indicates a non-responder. Hereinafter, the patients 201 and 202 who are responders are referred to as patients 201 (+) and 202 (+), and the patients 201 and 202 who are non-responders are referred to as patients 201 (−) and 202 (−).

That is, the patient 201 (+) is a patient whose injury or illness is cured by a procedure, and the patient 201 (−) is a patient whose injury or illness is not cured even when receiving the procedure. The patient 202 (+) is a patient whose injury or illness is cured even when receiving no procedure, and the patient 202 (−) is a patient whose injury or illness is not cured without a procedure. In FIG. 2, for simplicity of description, a set of six patients 201 and 202 is referred to as the population 200.

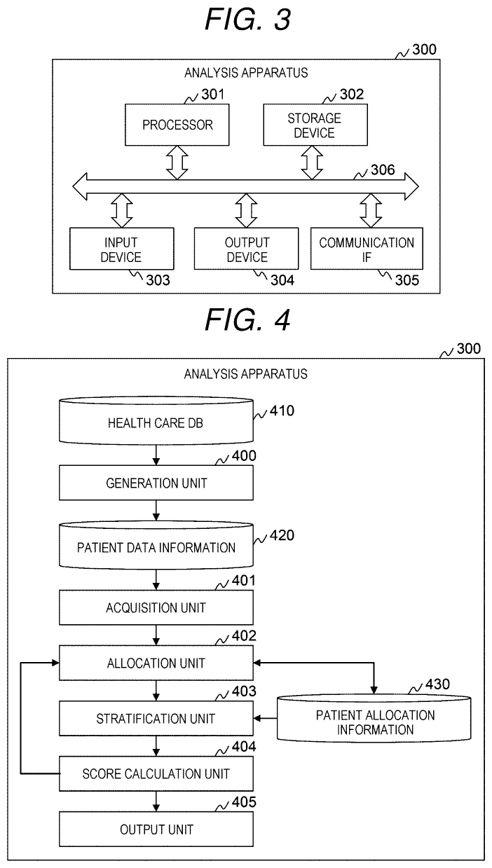

An analysis apparatus 300 (see FIG. 3) partitions the population 200 of patients into two subsets based on a predictive factor x in the patient characteristic considered to have a significant effect on the treatment effect τ. One of the subsets is referred to as a subtype L, and the other subset is referred to as a subtype R.

An estimated treatment effect τ(L) of the subtype L is a difference between an outcome of the patient 201 (+) in the subtype L and an outcome of the patient 202 (−) in the subtype L, and corresponds to the difference in the treatment effect τ between the patient groups C and D in FIG. 1.

An estimated treatment effect τ(R) of the subtype R is a difference between an outcome of the patients 201 (+) and 201 (−) in the subtype R and an outcome of the patient 202 (+) in the subtype R, and corresponds to the difference in the treatment effect τ between the patient groups C and D in FIG. 1.

The analysis apparatus trains a loss function f using a sum of squares of the estimated treatment effects τ(L) and τ(R) (formula (1) below), or predicts the treatment effect τ of a patient to be predicted by the loss function f.

$$\begin{matrix}Formula 1 & \\ f = {\sum }_{l}^{L , R} \{ N ( l ) \cdot {τ ( l )}^{2} \} & ( 1 )\end{matrix}$$

Here, l is an index indicating whether a treatment effect τ(l) is of the subtype L or R. In addition, N(l) is the number of samples of the subtype L.

### First Embodiment

FIG. 3 is a block diagram showing an example of a hardware structure of the analysis apparatus according to a first embodiment.

The analysis apparatus 300 includes a processor 301, a storage device 302, an input device 303, an output device 304, and a communication interface (communication IF) 305. The processor 301, the storage device 302, the input device 303, the output device 304, and the communication IF 305 are connected to one another via a bus 306.

The processor 301 controls the analysis apparatus 300. The storage device 302 is a work area of the processor 301. The storage device 302 is a non-transitory or transitory recording medium that stores various programs and data. Examples of the storage device 302 include a read only memory (ROM), a random access memory (RAM), a hard disk drive (HDD), and a flash memory. The input device 303 inputs data. Examples of the input device 303 include a keyboard, a mouse, a touch panel, a numeric keypad, a scanner, a microphone, and a sensor. The output device 304 outputs data. Examples of the output device 304 include a display, a printer, and a speaker. The communication IF 305 is connected to a network to transmit and receive data.

A function of the analysis apparatus 300 may be implemented using a computer system including a plurality of computers. The function of the analysis apparatus 300 may also be implemented using virtualization technology.

FIG. 4 is a block diagram showing an example of a functional configuration of the analysis apparatus in the first embodiment.

The analysis apparatus 300 includes a generation unit 400, an acquisition unit 401, an allocation unit 402, a stratification unit 403, a score calculation unit 404, and an output unit 405. The analysis apparatus 300 also retains a health care DB 410, patient data information 420, and patient allocation information 430.

The health care DB 410, the patient data information 420, and the patient allocation information 430 are stored in the storage device 302 and can be accessed by the processor 301.

The health care DB 410 stores health care data including a factor representing the characteristic of the patient as a field. A specific data structure will be described later. The patient data information 420 stores patient data shaped for data processing. A specific data structure will be described later. The patient allocation information 430 is information for managing an allocation of two data sets of patients (patient data) in stratification processing.

The generation unit 400, the acquisition unit 401, the allocation unit 402, the stratification unit 403, the score calculation unit 404, and the output unit 405 are functions implemented by the processor 301 executing a program stored in the storage device 302.

The generation unit 400 generates the patient data information 420 from the health care DB 410. The acquisition unit 401 acquires the patient data from the patient data information 420.

The allocation unit 402 allocates the patient data stored in the patient data information 420 to one of a first patient data set and a second patient data set. The first patient data set is a data set used for searching for a branching condition. The branching condition is a condition for partitioning a target patient group into two groups. The second patient data set is a data set used in calculation processing of evaluation metric for determining a treatment effect, that is, an intervention effect of a group partitioned based on the branching condition.

The stratification unit 403 repeats stratification of the patient data set and generates a decision tree. Specifically, the stratification unit 403 searches for the branching condition for the stratification of the data set, and repeatedly executes processing of partitioning the patient data set based on the discovered branching condition.

The score calculation unit 404 calculates a score for quantitatively evaluating quality of a branch of the decision tree generated by the stratification unit 403. The output unit 405 generates and outputs stratification information based on the decision tree and the score.

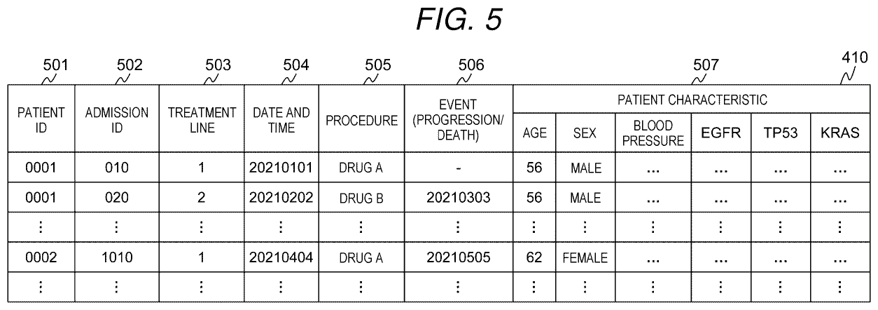

FIG. 5 shows an example of the health care DB 410 in the first embodiment.

The health care DB 410 stores an entry including a patient ID 501, an admission ID 502, a treatment line 503, a date 504, a procedure 505, an event 506, and a patient characteristic 507 as fields. One entry corresponds to one piece of health care data. There are one or more pieces of health care data for one patient. For example, in a case where a certain patient is admitted three times, three pieces of health care data of the patient are stored in the health care DB 410. In FIG. 5, health care data about injury or illness (for example, cancer) to be analyzed is defined.

The patient ID 501 is a field that stores identification information for uniquely identifying the patient. The admission ID 502 is a field that stores identification information allocated when the patient is admitted.

The treatment line 503 is a field that stores a number indicating an order of treatments for the cancer (for example, administration of anticancer drugs). For example, when an anticancer drug is administered for a first time to a certain carcinoma, a value of the treatment line 503 is “1” for a first treatment, “2” for a second treatment, and “3” for a third treatment.

The date 504 is a field that stores date and time of the treatment (year, month, and day). The procedure 505 is a field that stores a content of the treatment. The event 506 is a field that stores a result of the treatment (for example, progression or death).

The patient characteristic 507 is a field that stores a value of the factor representing the characteristic of the patient at the date and time stored in the date 504. The factor includes a covariate. The patient characteristic 507 includes, for example, an age, a sex, blood pressure, EGFR, TP53, and KRAS.

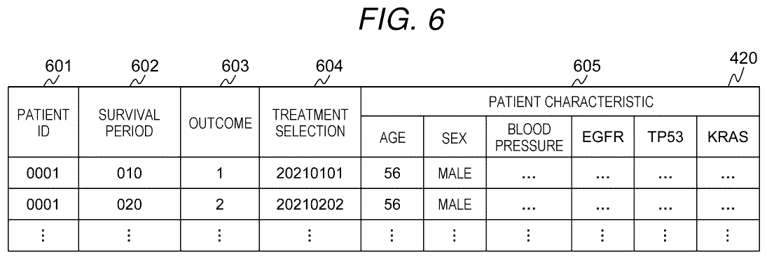

FIG. 6 shows an example of the patient data information 420 in the first embodiment.

The patient data information 420 stores the patient data in which the health care data stored in the health care DB 410 is collected in units of patients. The patient data includes a patient ID 601, a survival period 602, an outcome 603, a treatment selection 604, and a patient characteristic 605 as fields. One entry corresponds to patient data of one patient.

When there are a plurality of pieces of health care data of the same patient in the health care DB 410, for example, health care data in which the treatment line 503 has a maximum value is shaped as the patient data.

The patient ID 601 and the patient characteristic 605 are fields identical to the patient ID 501 and the patient characteristic 507.

The survival period 602 is a field that stores a period from when the patient receives the treatment to when the patient dies. The survival period 602 stores a period determined by the date and time of the date 504 and date and time of the event 506. When there is no value in the event 506, a period determined by the date and time of the date 504 and current date and time is stored.

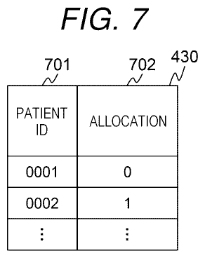

The outcome 603 is, for example, an observed value such as survival, progression-free survival, or a tumor size, and is a field that stores a value inherently including a non-treatment-related effect and a treatment effect. In FIG. 7, a numerical value indicating survival is stored in the outcome 603. For example, “1” indicates survival and “0” indicates death. The analysis apparatus 300 refers to the event 506, and stores “1” when no date of death is stored in the event 506, and stores “0” when the date of death is stored in the event 506.

The treatment selection 604 is a field that stores a value indicating whether the patient selects the treatment. Here, “1” indicates that the patient selects the treatment, and “0” indicates that the patient does not select the treatment. The analysis apparatus 300 refers to the procedure 505, stores “0” when no value is stored in the procedure 505, and stores “1” when a value is stored in the procedure 505.

In the following description, for convenience of description, EGFR may be referred to as a factor x1, TP53 may be referred to as a factor x2, and KRAS may be referred to as a factor x3.

FIG. 7 shows an example of the patient allocation information 430 in the first embodiment.

The patient allocation information 430 stores an entry including a patient ID 701 and an allocation 702. The patient ID 701 is the same field as the patient ID 501.

The allocation 702 is a field that stores a value indicating which of the first patient data set and the second patient data set the patient is to be allocated. When the patient is to be allocated to the first patient data set, “0” is stored, and when the patient is to be allocated to the second patient data set, “1” is stored.

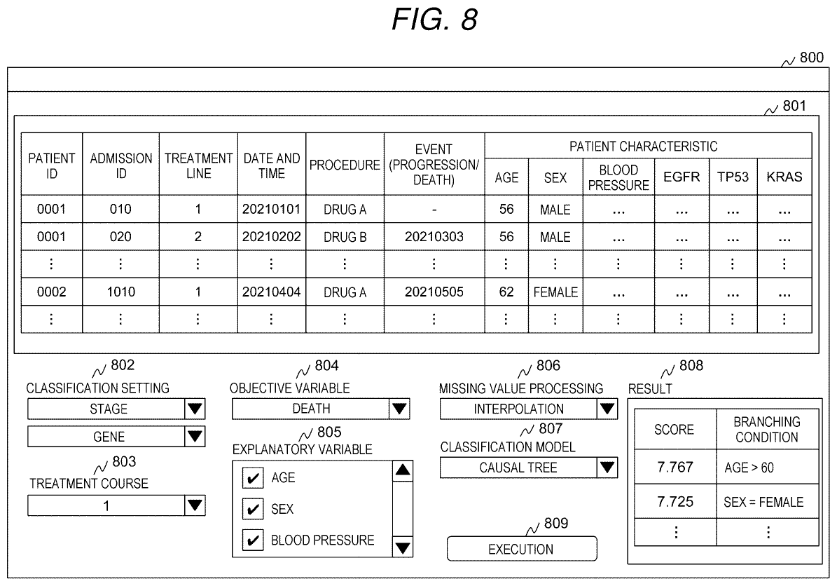

FIG. 8 shows an example of an input screen presented by the analysis apparatus 300 in the first embodiment.

An input screen 800 is displayed on a display apparatus, which is an example of the output device 304 of the analysis apparatus 300, or on a display apparatus of another computer that can communicate with the analysis apparatus 300 via the communication IF 305. A user can input information to the input screen 800 by operating the input device 303 of the analysis apparatus 300 or an input device of another computer.

The input screen 800 includes setting fields 801, 802, 803, 804, 805, 806, and 807, a display field 808, and an execution button 809.

The setting field 801 is a field for selecting an entry to be used for processing from the health care DB 410.

The setting field 802 is a field for selecting an item to be used for classifying a plurality of entries selected in the setting field 801. For example, it is conceivable that the classification is based on a stage or a gene of the cancer of the patient. By selecting the item, the entries can be narrowed down.

The setting field 803 is a field for selecting the treatment line of the patient. In the setting field 803, a value that can be set in the treatment line 503 is displayed in a pull-down format.

The setting field 804 is a field for selecting an objective variable output from a classification model f. As the objective variable, for example, an event or a procedure of the patient can be selected.

The setting field 805 is a field for selecting an explanatory variable of the patient. As the explanatory variable, a factor in the patient characteristic 507 can be selected. In FIG. 8, the age, the sex, and the blood pressure are selected.

The setting field 806 is a field for selecting missing value processing of the explanatory variable. In FIG. 8, “interpolation” is selected as the missing value processing.

The setting field 807 is a field for selecting the classification model f. In FIG. 8, a causal tree is selected as the classification model f. The causal tree is a type of a decision tree for calculating conditional average treatment effect (CATE) in statistical causal inference.

The display field 808 is a field for displaying the decision tree and the score. In FIG. 8, a pair of a decision tree branching condition group and the score is displayed.

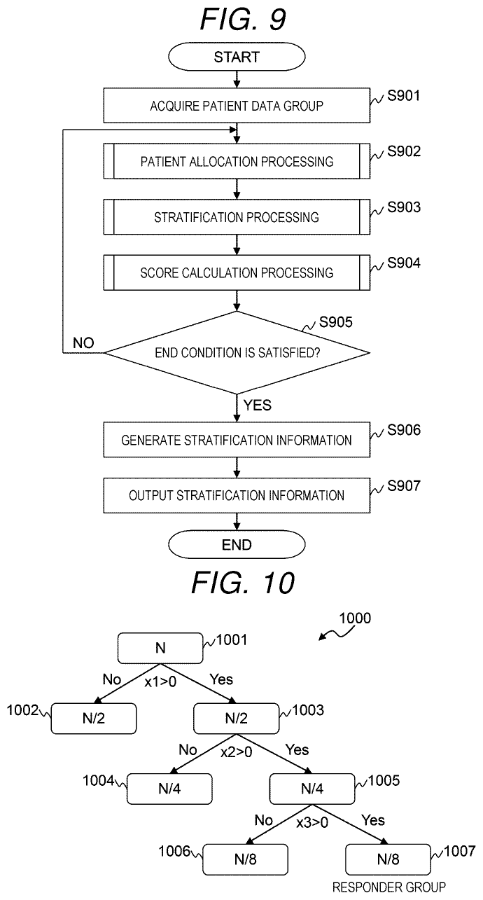

FIG. 9 is a flowchart showing an example of analysis processing executed by the analysis apparatus 300 in the first embodiment.

Here, it is assumed that the generation unit 400 of the analysis apparatus 300 generates the patient data information 420.

The acquisition unit 401 of the analysis apparatus 300 acquires the patient data from the patient data information 420 (step S901).

Next, the allocation unit 402 of the analysis apparatus 300 executes patient allocation processing (step S902). In the patient allocation processing, patients are allocated to the first patient data set and the second patient data set. Details of the patient allocation processing will be described later.

Next, the stratification unit 403 of the analysis apparatus 300 executes the stratification processing (step S903). Details of the stratification processing will be described later.

Next, the score calculation unit 404 of the analysis apparatus 300 executes score calculation processing on a causal tree generated in the stratification processing (step S904). Details of the score calculation processing will be described later.

Next, the analysis apparatus 300 determines whether an end condition is satisfied (step S905). For example, when the number of times of execution of a series of processing of the patient allocation processing, the stratification processing, and the score calculation processing is larger than a predetermined value, the analysis apparatus 300 determines that the end condition is satisfied.

When the end condition is not satisfied, the analysis apparatus 300 returns to step S902 and executes the same processing.

When the end condition is satisfied, the output unit 405 of the analysis apparatus 300 generates the stratification information (step S906). For example, the output unit 405 generates a list in which causal trees are sorted in a descending order of scores as the stratification information. The output unit 405 also generates a list of a causal tree having a maximum score as the stratification information.

The output unit 405 of the analysis apparatus 300 outputs the stratification information (step S907) and ends the analysis processing.

For example, the output unit 405 of the analysis apparatus 300 may display the stratification information on a display, which is an example of the output device 304, may transmit the stratification information to another computer by the communication IF 305, or may store the stratification information in the storage device 302.

FIG. 10 shows an example of the causal tree generated by the stratification processing executed by the analysis apparatus 300 in the first embodiment.

In the stratification processing, a causal tree 1000 as shown in FIG. 10 is generated. The causal tree 1000 includes nodes 1001 to 1007. Here, “N” in the nodes 1001 to 1007 represents the number of samples, that is, the number of patients (patient data). The causal tree 1000 has a tree structure in which the number of samples is halved by partitioning.

A patient group indicated by the node 1001 is partitioned into a patient group (node 1003) in which the factor x1 is larger than 0 and a patient group (node 1002) in which the factor x1 is 0 or less. A formula determined by the factor x1 and the threshold “0” is a branching condition of the node 1001.

The patient group corresponding to the node 1003 is partitioned into a patient group (node 1005) in which the factor x2 is larger than 0 and a patient group (node 1004) in which the factor x2 is 0 or less. A formula determined by the predictive factor x2 and the threshold “0” is a branching condition of the node 1003.

The patient group corresponding to the node 1005 is partitioned into a patient group (node 1007) in which the factor x3 is larger than 0 and a patient group (node 1006) in which the factor x3 is 0 or less. A formula determined by the factor x3 and the threshold “0” is a branching condition of the node 1005. The node 1007 is a responder group in the causal tree 1000.

There is no branching condition in the nodes 1002, 1004, 1006, and 1007. The nodes 1001 to 1007, a connection relationship between the nodes 1001 to 1007, and the branching conditions of the nodes 1001, 1003, and 1005 are retained as information constituting the causal tree 1000.

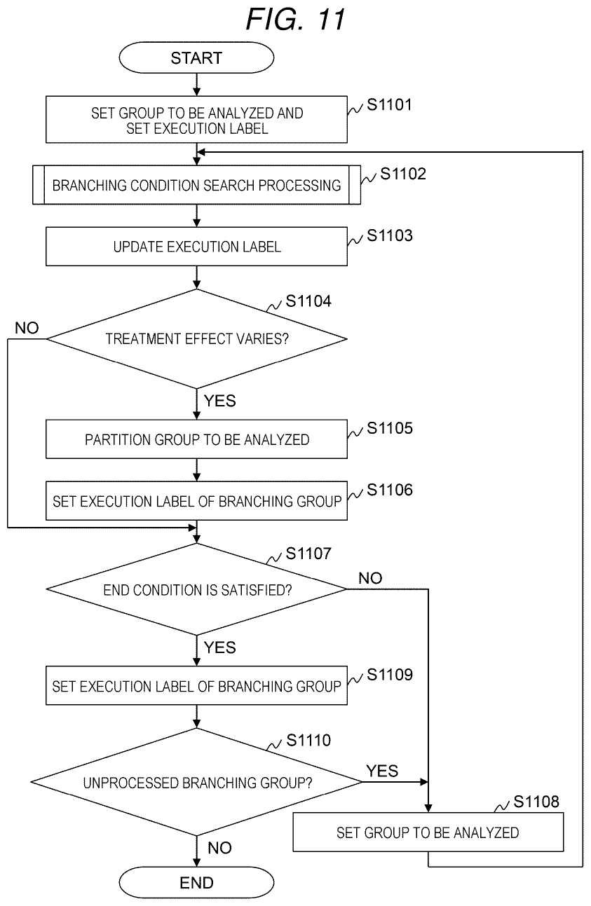

FIG. 11 is a flowchart showing an example of the stratification processing executed by the analysis apparatus 300 in the first embodiment.

The stratification unit 403 sets a plurality of pieces of patient data acquired by the acquisition unit 401 as a group to be analyzed, and sets an execution label [K, V] that is a combination of a key K and a value V (step S1101).

The stratification unit 403 sets the value V to “False” indicating that the branching condition search processing is not executed, and sets the key K to “1”.

Next, the stratification unit 403 executes the branching condition search processing (step S1102). Details of the branching condition search processing will be described later.

Next, the stratification unit 403 updates the execution label [K, V] (step S1103). Specifically, the stratification unit 403 updates the value V to “True”.

Next, the stratification unit 403 determines whether the treatment effect varies before and after partitioning the group to be analyzed (step S1104).

Specifically, the stratification unit 403 partitions, based on the branching condition obtained from the branching condition search processing, the group to be analyzed, and generates a first branching group and a second branching group. At this stage, a result of partitioning is not reflected. The analysis apparatus 300 determines which of the first branching group and the second branching group has a treatment effect that significantly varies with respect to a treatment effect of the group to be analyzed.

For example, the stratification unit 403 calculates a standard deviation obtained by combining a treatment effect difference obtained by comparing the first branching group with the group to be analyzed (hereinafter referred to as a first difference) and a treatment effect difference obtained by comparing the second branching group with the group to be analyzed (hereinafter referred to as a second difference). The stratification unit 403 determines whether at least one of the first difference and the second difference is larger than the standard deviation. When at least one of the first difference and the second difference is larger than the standard deviation, it is determined that the treatment effect varies before and after partitioning the group to be analyzed.

When None is output as a result of the branching condition search processing, the stratification unit 403 does not perform the above-described processing, and determines that the treatment effect does not vary before and after partitioning the group to be analyzed.

When it is determined that the treatment effect does not vary before and after partitioning the group to be analyzed, the stratification unit 403 proceeds to step S1107.

When it is determined that the treatment effect varies before and after partitioning the group to be analyzed, the stratification unit 403 partitions the group to be analyzed based on the branching condition used in step S1104 (step S1105).

The stratification unit 403 sets an execution label in each of the first branching group and the second branching group (step S1106), and then proceeds to step S1107. Specifically, the following processing is executed.

(S1106-1) The stratification unit 403 generates two copies of the execution label [K, V] of the group to be analyzed.

(S1106-2) The stratification unit 403 appends a branching number “1” to an end of the key K of the execution label [K, V] and sets the execution label whose value V is changed from V=True to V=False as the execution label of the first branching group. Similarly, the stratification unit 403 appends a branching number “2” to the end of the key K of the execution label [K, V] and sets the execution label whose value V is changed from V=True to V=False as the execution label of the second branching group.

For example, when the execution label of the group to be analyzed is [1, True], the execution label of the first branching group is [11, False], and the execution label of the second branching group is [12, False].

In step S1107, the stratification unit 403 determines whether the end condition is satisfied (step S1107). The end condition is set, for example, based on the number of times of partitioning (branching depth) or the number of samples in the group. Specifically, when the number of times of partitioning is equal to or larger than a threshold, it is determined that the end condition is satisfied. When the number of samples of any branching group after partitioning is smaller than a threshold, it is determined that the end condition is satisfied.

When it is determined that the end condition is not satisfied, the stratification unit 403 selects one branching group from the first branching group and the second branching group obtained by partitioning the group to be analyzed, sets the selected branching group as a new group to be analyzed (step S1108), and then returns to step S1102.

When it is determined that the end condition is satisfied, the stratification unit 403 updates the execution labels of the first branching group and the second branching group (step S1109). Specifically, the stratification unit 403 updates the value V of the execution label of each of the first branching group and the second branching group to “True”. When there is no branching condition, the processing of step S1109 is skipped.

Next, the stratification unit 403 determines whether there is any unprocessed branching group (step S1110). Specifically, it is determined whether there is a branching group in which the value V of the execution label is “False”. When there is a branching group in which the value V of the execution label is “False”, it is determined that there is an unprocessed branching group.

When there are unprocessed branching groups, the stratification unit 403 selects one branching group from the unprocessed branching groups, sets the selected branching group as a new group to be analyzed (step S1108), and then returns to step S1102.

When there is no unprocessed branching group, the stratification unit 403 ends the stratification processing. Here, the stratification processing will be described using the causal tree 1000 shown in FIG. 10 as an example. At a first time, a set of a plurality of pieces of patient data acquired by the acquisition unit 401 is set as the group to be analyzed (step S1101). The set of patient data corresponds to the node 1001. In step S1104, the stratification unit 403 partitions, based on a branching condition (x1>0), the group to be analyzed into the first branching group (x1>0: YES) and the second branching group (x1>0: NO), and determines whether the treatment effect varies. Here, it is assumed that the treatment effect varies for one of the first branching group (x1>0: YES) and the second branching group (x1>0: NO). In this case, the stratification unit 403 partitions the group to be analyzed into the first branching group (x1>0: NO) and the second branching group (x1>0: YES) under the branching condition (x1>0) (step S1105). The stratification unit 403 sets the execution label [11, False] of the first branching group (x1>0: NO) and the execution label [12, False] of the second branching group (x1>0: YES) using the execution label [1, True] of the group to be analyzed (step S1106). The first branching group (x1>0: NO) corresponds to the node 1002, and the second branching group (x1>0: YES) corresponds to the node 1003.

In step S1108, the stratification unit 403 sets the first branching group (x1>0: NO) as the group to be analyzed. The stratification unit 403 executes the branching condition search processing on the first branching group (x1>0: NO) (step S1102) and updates the execution label to [11, True] (step S1103). Here, since there is no branching condition for the first branching group (x1>0: NO), the stratification unit 403 ends the search for the first branching group (x1>0: NO) (step S1104: NO).

In step S1110, since the execution label of the second branching group (x1>0: YES) is [12, False], the stratification unit 403 determines that there is an unprocessed branching group (step S1110: YES), and sets the second branching group (x1>0: YES) as the group to be analyzed (step S1108).

The stratification unit 403 executes the branching condition search processing on the group to be analyzed (x1>0: YES) (step S1102) and updates the execution label to [12, True] (step S1103). In step S1104, the stratification unit 403 partitions, based on a branching condition (x2>0), the group to be analyzed (x1>0: YES) into a third branching group (x2>0: NO) and a fourth branching group (x2>0: YES). Here, it is assumed that the treatment effect varies for one of the third branching group (x2>0: NO) and the fourth branching group (x2>0: YES). In this case, the stratification unit 403 partitions the group to be analyzed (x1>0: YES) into the third branching group (x2>0: NO) and the fourth branching group (x2>0: YES) under the branching condition (x2>0) (step S1105). The stratification unit 403 sets an execution label [123, False] of the third branching group (x2>0: NO) and an execution label [124, False] of the fourth branching group (x2>0: YES) using the execution label [12, True] of the group to be analyzed (step S1106). The third branching group (x2>0; NO) corresponds to the node 1004, and the fourth branching group (x2>0: YES) corresponds to the node 1005.

In step S1108, the stratification unit 403 sets the third branching group (x2>0: NO) as the group to be analyzed. The stratification unit 403 executes the branching condition search processing on the third branching group (x2>0: NO) (step S1102) and updates the execution label to [123, True] (step S1103). Here, since there is no branching condition for the third branching (x2>0: NO), the group stratification unit 403 ends the search for the third branching group (x2>0: NO) (step S1104: NO).

In step S1110, since the execution label of the fourth branching group (x2>0: YES) is [124, False], the stratification unit 403 determines that there is an unprocessed branching group (step S1110: YES), and sets the fourth branching group (x2>0: YES) as the group to be analyzed (step S1108).

The stratification unit 403 executes the branching condition search processing on the group to be analyzed (x2>0: YES) (step S1102) and updates the execution label to [124, True] (step S1103). In step S1104, the stratification unit 403 partitions, based on a branching condition (x3>0), the group to be analyzed (x2>0: YES) into a fifth branching group (x3>0: NO) and a sixth branching group (x3>0: YES). Here, it is assumed that the treatment effect varies for one of the fifth branching group (x3>0: NO) and the sixth branching group (x3>0: YES). In this case, the stratification unit 403 partitions the group to be analyzed (x2>0: YES) into the fifth branching group (x3>0: NO) and the sixth branching group (x3>0: YES) under the branching condition (x3>0) (step S1105). The stratification unit 403 sets an execution label [1245, False] of the fifth branching group (x3>0: NO) and an execution label [1246, False] of the sixth branching group (x3>0: YES) using the execution label [124, True] of the group to be analyzed (step S1106). The fifth branching group (x3>0: NO) corresponds to the node 1006, and the sixth branching group (x3>0: YES) corresponds to the node 1007.

In step S1108, the stratification unit 403 sets the fifth branching group (x3>0: NO) as the group to be analyzed. The stratification unit 403 executes the branching condition search processing on the fifth branching group (x3>0: NO) (step S1102) and updates the execution label to [1245, True] (step S1103). Here, since there is no branching condition for the fifth branching group (x3>0: NO), the stratification unit 403 ends the search for the fifth branching group (x3>0: NO) (step S1104: NO).

In step S1110, since the execution label of the sixth branching group (x3>0: YES) is [1246, False], the stratification unit 403 determines that there is an unprocessed branching group (step S1110: YES), and sets the sixth branching group (x3>0: YES) as the group to be analyzed (step S1108).

The stratification unit 403 executes the branching condition search processing on the sixth branching group (x3>0: YES) (step S1102) and updates the execution label to [1246, True] (step S1103). Here, since there is no branching condition for the sixth branching group (x3>0: YES), the stratification unit 403 ends the search for the sixth branching group (x3>0: YES) (step S1104: NO).

In step S1110, since there is no branching group whose value V is “False”, the stratification unit 403 ends the stratification processing. The stratification unit 403 outputs the branching groups, the execution labels, and the branching conditions as a processing result.

In this way, in the stratification processing, a search is executed to maximize the treatment effect for each branching group generated by branching.

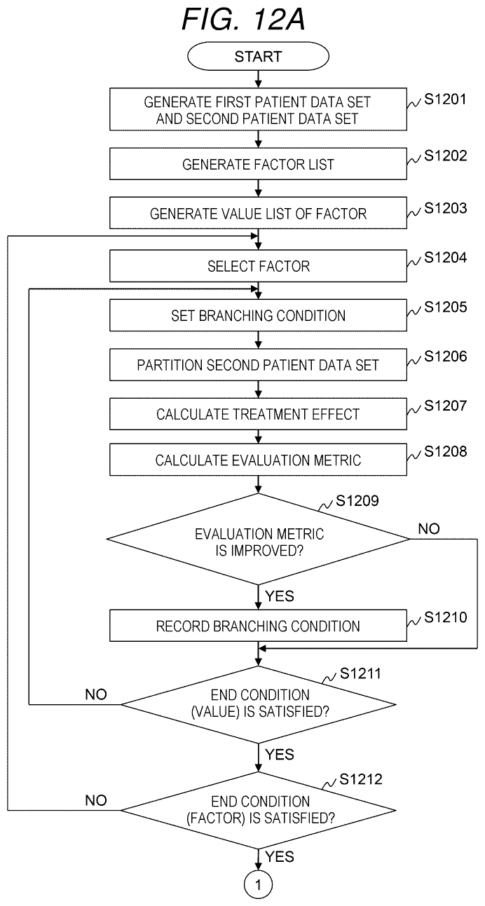

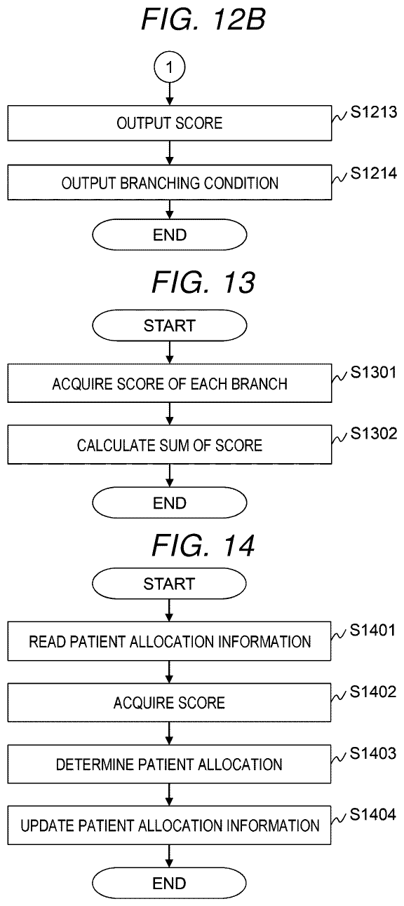

FIGS. 12A and 12B are flowcharts showing an example of the branching condition search processing executed by the analysis apparatus 300 in the first embodiment.

The stratification unit 403 refers to the patient allocation information 430 and generates the first patient data set and the second patient data set from the group to be analyzed set in step S1101 or step S1108 (step S1201).

Next, the stratification unit 403 generates a list of factors (factor list) by randomly selecting factors that are covariates in the patient data (step S1202). The factor list is a list of the age, the blood pressure, EGFR, and the like. A causal tree is created for each factor list.

Next, the stratification unit 403 refers to the first patient data set and generates a value list of the selected factors (step S1203). The value list of the factors is a list of ages such as 56 years old and 62 years old when the factor is the age, and is a list of blood pressure values such as 90 ml and 127 ml when the factor is the blood pressure.

Next, the stratification unit 403 selects a factor from the factor list (step S1204).

Next, the stratification unit 403 refers to the value list of the selected factors to set the branching condition (step S1205).

Next, the stratification unit 403 partitions the second patient data set into two branching groups based on the branching condition (step S1206). Here, one of the partitioned branching groups is referred to as the subtype L, and the other branching group is referred to as the subtype R.

Next, stratification unit 403 calculates the treatment effect τ of each of the subtypes L and R (step S1207). The treatment effect τ is calculated using formula (2), for example.

$$\begin{matrix}Formula 2 & \\ τ ( l ) = E [ Y \vert T = 1 ] - E [ Y \vert T = 0 ] & ( 2 )\end{matrix}$$

Here, l is a variable representing a branching group, l=L in the case of the subtype L, and l=R in the case of the subtype R. In addition, Y is a variable representing the outcome (for example, an event 606). In addition, T is a binary variable indicating the treatment selection, T=1 indicates that the treatment is selected, and T=0 indicates that no treatment is selected. In addition, E [ ] is an expectation operator. Here, E [ ] is, for example, a sum of an outcome Y. The treatment effects τ(L) and τ(R) are both expressed as τ(l) when not distinguished.

Next, the stratification unit 403 calculates an evaluation metric of the branching condition using the treatment effects τ(L) and τ(R) (step S1208). Specifically, the following processing is executed.

(S1208-1) The stratification unit 403 executes calculation of a loss function LossPre before partitioning represented by formula (3).

$$\begin{matrix}Formula 3 & \\ LossPre = N \cdot τ & ( 3 )\end{matrix}$$

Here, N is the number of samples in a group to be searched. In addition, τ is a treatment effect in a patient data set before partitioning. During first execution, the treatment effect τ in a parent node is used.

The loss function LossPre defined by formula (4) obtained by adding a penalty term for a variance to formula (3) may be used.

$$\begin{matrix}Formula 4 & \\ LossPre = N \cdot τ - ( 1 + \frac{{N}_{train}}{{N}_{est}} ) * ( \frac{{S}_{T = 1}^{2}}{p} + \frac{{S}_{T = 0}^{2}}{1 - p} ) & ( 4 )\end{matrix}$$

Here, Ntrain is the number of samples in the first patient data set. In addition, Nest is the number of samples in the second patient data set. In addition, ST=1 is a variance of samples belonging to the treatment selection T=1 in the first patient data set, and ST=0 is a variance of samples belonging to the treatment selection T=0 in the first patient data set. In addition, p is a ratio of the number of samples belonging to the treatment selection T=1 in the first patient data set.

An entire right-hand side of each of the formulas (3) and (4) may be divided by the number of samples N in the first patient data set and thus be normalized.

(S1208-2) The stratification unit 403 executes calculation of a loss function LossPost after partitioning represented by formula (5).

$$\begin{matrix}Formula 5 & \\ LossPost = {\sum }_{l}^{L , R} {N}_{l} \cdot {τ}_{l} & ( 5 )\end{matrix}$$

Here, N(l) is the number of samples of a subtype l. When the loss function LossPre is normalized, an entire right-hand side of the formula (5) may be divided by the number of samples in the first patient data set and thus be normalized.

(S1208-3) The stratification unit 403 calculates a difference Gain between the loss function LossPre and the loss function LossPost as the evaluation metric. The difference Gain is a metric indicating whether the loss function LossPost is improved by partitioning.

Next, the stratification unit 403 determines whether the evaluation metric is improved (step S1209). Specifically, the stratification unit 403 determines whether the current difference Gain is larger than a retained difference Gain. When the current difference Gain is larger than the retained difference Gain, the stratification unit 403 determines that the evaluation metric is improved.

Here, the retained difference Gain is the difference Gain recorded in previous loop processing and is a target value. However, since there is no difference Gain during the first execution, 0 is used as an initial value of the difference Gain being retained.

When it is determined that the evaluation metric is not improved, the stratification unit 403 proceeds to step S1211.

When it is determined that the evaluation metric is improved, the stratification unit 403 records the branching condition (step 1210). At this time, the stratification unit 403 updates the retained difference Gain with the current difference Gain.

Next, the stratification unit 403 determines whether a value end condition is satisfied (step S1211). The value end condition is, for example, a case where there is no selectable value.

When the value end condition is satisfied, the stratification unit 403 determines whether a factor end condition is satisfied (step S1212). The factor end condition is, for example, a case where there is no selectable factor.

When no end condition is satisfied, the stratification unit 403 returns to step S1204. When the end condition is satisfied, the stratification unit 403 calculates a score from a metric for determining a significant difference when the data set is partitioned based on the recorded branching condition (step S1213). For example, the metric for determining the significant difference is a p-value, and a sum of base-10 logarithms of p-values of each partitioned data set is calculated as the score.

Next, the stratification unit 403 outputs the branching condition (step S1214) and then ends the branching condition search processing. At this time, the stratification unit 403 also outputs the score.

FIG. 13 is a flowchart showing an example of the score calculation processing executed by the analysis apparatus 300 in the first embodiment.

The stratification unit 403 acquires the score of each branch of the causal tree (step S1301). The score is a value calculated in the branching condition search processing.

The stratification unit 403 calculates a sum of the score (step S1302). A sum of scores of respective branching conditions is a final score of the causal tree.

The method for calculating the score described with reference to FIG. 13 is merely an example, and the method is not limited thereto.

FIG. 14 is a flowchart showing an example of the patient allocation processing executed by the analysis apparatus 300 in the first embodiment.

Generally, an allocation of patients to the first patient data set and the second patient data set is performed randomly. In the present embodiment, the allocation of patients is treated as an optimization problem. Specifically, the allocation of patients is determined to maximize a desired objective function. By optimizing the allocation of patients, a causal tree with high interpretability can be generated. Hereinafter, an example of the patient allocation processing will be described.

The allocation unit 402 reads the patient allocation information 430 (step S1401). At a first time, the allocation unit 402 sets a random allocation result in the patient allocation information 430.

The allocation unit 402 acquires a score of a causal tree generated in previous loop processing (step S1402). At the first time, a preset initial score is acquired. The initial score is, for example, 0.

Next, the allocation unit 402 sets the score as the objective function, and determines the allocation of patients to each data set using a metaheuristic optimization method that maximizes the score (step S1403). The metaheuristic optimization method is, for example, a genetic algorithm.

Next, the allocation unit 402 updates the patient allocation information 430 based on a processing result in step S1403 (step S1404).

According to the present embodiment, the analysis apparatus 300 can show a quantitative evaluation of prediction accuracy of the treatment effect. It is also possible to generate a causal tree with higher accuracy by optimizing the allocation of patients in the stratification processing.

As described above, according to the analysis apparatus 300, the causal tree is generated from the analysis target, which is the original population.

In the embodiment, the patient data is described as an example of data to be analyzed, and the data to be analyzed may be any data that can be generated by the causal tree, and a type and a content of the data to be analyzed are not limited.

The invention is not limited to the embodiment described above, and includes various modifications. For example, the embodiment described above is described in detail to facilitate understanding of the invention, and the invention is not necessarily limited to those including all the described configurations. A part of a configuration in each embodiment may be added to, deleted from, or replaced with another configuration.

A part or all of configurations, functions, processing units, processing methods, and the like described above may be implemented by hardware by, for example, designing with an integrated circuit. The invention can also be implemented by a program code of software for implementing the functions of the embodiment. In this case, a storage medium storing the program code is provided to a computer, and a processor provided in the computer reads the program code stored in the storage medium. In this case, the program code read from the storage medium implements the functions of the embodiment described above by itself, and the program code itself and the storage medium storing the program code implement the invention. Examples of the storage medium for supplying such a program code include a flexible disk, a CD-ROM, a DVD-ROM, a hard disk, a solid state drive (SSD), an optical disk, a magneto-optical disk, a CD-R, a magnetic tape, a non-volatile memory card, and a ROM.

The program code for implementing the functions described in the embodiment can be implemented in a wide range of programs or script languages such as Assembler, C/C++, perl, Shell, PHP, Python, and Java (registered trademark).

Further, the program code of software for implementing the functions of the embodiment may be distributed via a network to be stored in a storage unit such as a hard disk or a memory of a computer or a storage medium such as a CD-RW or a CD-R, and a processor provided in the computer may read and execute the program code stored in the storage unit or the storage medium.

Control lines and information lines considered to be necessary for description are shown in the embodiment described above, and not all control lines and information lines are necessarily shown in a product. All the configurations may be connected.

What is claimed is:

1. A computer system comprising:
a processor; and
a storage apparatus connected to the processor, wherein
the computer system is accessibly connected to a database that stores data for evaluating an intervention effect, the data including values of a plurality of factors, and
the processor
repeatedly executes: first processing of partitioning an analysis data set including a plurality of pieces of the data into a first data set and a second data set; second processing of searching, using the first data set, for a branching condition for partitioning the analysis data set into two groups, the branching condition being defined by the factors and the values of the factors, evaluating the intervention effect using the second data set, determining the branching condition to be used, and generating a decision tree that includes at least one branching condition and is used to predict an event; and third processing of calculating a score indicating quality of a branch of the decision tree for each of a plurality of the decision trees, and
generates and outputs information for displaying the plurality of decision trees and the score.

2. The computer system according to claim 1, wherein
the data includes a value indicating whether the event occurs,
in the second processing, the processor calculates an evaluation metric for evaluating presence or absence of a statistically significant difference when the analysis data set is partitioned based on the branching condition, and calculates the score based on the evaluation metric, and
in the third processing, the processor calculates a sum of the score of the branching condition in the decision tree.

3. The computer system according to claim 2, wherein
the processor generates the information for displaying the decision tree whose sum of the score is maximized.

4. The computer system according to claim 1, wherein
in the first processing, the processor determines an allocation of the data constituting the analysis data set for the first data set and the second data set by setting the score as an objective variable and executing a computation of a metaheuristic optimization method to optimize the score.

5. The computer system according to claim 1, wherein
the data is data including a value of a factor indicating a characteristic of a patient.

6. A data analysis method executed by a computer system, wherein
the computer system
includes a processor and a storage apparatus connected to the processor, and
is accessibly connected to a database that stores data for evaluating an intervention effect, the data including values of a plurality of factors, and
the data analysis method comprises
a first step in which the processor repeatedly executes: first processing of partitioning an analysis data set including a plurality of pieces of the data into a first data set and a second data set; second processing of searching, using the first data set, for a branching condition for partitioning the analysis data set into two groups, the branching condition being defined by the factors and the values of the factors, evaluating the intervention effect using the second data set, determining the branching condition to be used, and generating a decision tree that includes at least one branching condition and is used to predict an event; and third processing of calculating, using the second data set, a score indicating quality of a branch of the decision tree for each of a plurality of the decision trees, and
a second step in which the processor generates and outputs information for displaying the plurality of decision trees and the score.
